(19) United States
(12) Patent Application Publication
(10) Pub. No.: US 2015/0125558 A1
(43) Pub. Date: May 7, 2015

(54) PLEUROPTERUS MULTIFLORUS EXTRACT AND DIPSACUS ASPEROIDES EXTRACT FOR SECRETING INSULIN-LIKE GROWTH FACTOR AND PROMOTING BONE STRUCTURE GROWTH, AND METHOD FOR PREPARING SAME

(71) Applicant: Sangkeun Hong, Seoul (KR)

(72) Inventor: Sangkeun Hong, Seoul (KR)

(21) Appl. No.: 14/394,368

(22) PCT Filed: Nov. 30, 2012
(86) PCT No.: PCT/KR2012/010274

§ 371 Date: Oct. 14, 2014

(30) Foreign Application Priority Data
Jun. 28, 2012 (KR) 10-2012-0070146

(51) Int. Cl. A61K 36/27; A23L 1/30; A61K 36/53

(52) U.S. Cl. CPC A61K 36/27; A61K 36/53; A23L 1/3002; A23V 2002/00
USPC 424/745; 424/725; 435/99

(57) ABSTRACT

The present invention relates to a purified aqueous extract material and to a method for preparing same, wherein the purified aqueous extract material includes, as a major ingredient, a glycoside ingredient of Pleuropterus multiflorus and Dipsacus asperoides which have the effect of increasing the secretion of an insulin-like growth factor and promoting bone structure growth.

### TECHNICAL FIELD

The present invention relates, in general, to an aqueous purified extract containing as active ingredients glycoside components of Pleuropterus multiflorus and Dipsacus asperoides; capable of increasing the secretion of an insulin-like growth factor and promoting bone structure growth; and a method of manufacturing the same.

### BACKGROUND ART

Generally, growth hormone is a protein secreted by the anterior lobe of the pituitary gland in humans and animals. In humans, growth hormone consists of 191 amino acids, and the secretion of growth hormone requires stimulation of the pituitary gland by a growth hormone-releasing hormone (GHRH), which is a substance that promotes the secretion of growth hormone.

A major role of growth hormone within a living subject is the production of insulin-like growth factor-1 (IGF-1) in peripheral tissues such as the liver. IGF-1 stimulates growth by acting on cartilage tissues and also increases protein synthesis via metabolic activities, thus increasing muscle mass and oxidation of fatty acids in adipose tissues, leading to reduction in body fat.

In this regard, growth hormone has long been used for treating dwarfism patients, and recently its potential use as an obesity treatment has been studied. In the past, patients treated with growth hormone obtained from pituitary gland of cadavers often developed Creutzfeldt-Jakob disease. Growth hormone has been commercially available since the late 1985, and mass-produced and supplied owing to the development of a genetic recombination technology. With the intent of reducing the risk of infecting patients with other diseases during growth hormone treatment, the growth hormone manufactured by the genetic recombination technology has been used as the sole treatment for treating growth hormone deficient patients.

The growth hormone manufactured by genetic recombination has an enormous protein structure and is thus administered via intramuscular injection instead of oral administration. Even with mass production, this type of growth hormone treatment requires an extremely high expense and is also considered inconvenient because it is administered via intra-muscular injection.

Korean Patent Application Publication No. 2002-46923 discloses a pharmaceutical composition comprising a pharmaceutically effective amount of Phlomis umbrosa Turez extract and a pharmaceutically acceptable carrier, for inducing IGF-1 secretion, wherein Phlomis umbrosa Turez extract is obtained by conducting a hot water extraction of Phlomis umbrosa Turez, followed by filtrating the resulting hot water extract via an ultrafiltration membrane with a cut-off of 30,000-100,000.

Korean Patent Application Publication No. 2003-22655 discloses a pharmaceutical composition comprising (a) a pharmaceutically effective amount of Cynachum wilfordii (Max) Hem & ley extract, a Zingiberis officinale and (b) a pharmaceutically acceptable carrier, for treating or preventing diseases induced by the reduction in the amount of IGF-1.

However, the IGF-1 secretion capacity of the pharmaceutical compositions described above appears insufficient to meet the commercial requirement and there is a need for the development of a pharmaceutical composition with an improved secretion capacity.

### DISCLOSURE

### Technical Problem

Accordingly, the present invention has been made keeping in mind the above problems occurring in the prior art, and an object of the present invention is to provide a method for manufacturing a Pleuropterus multiflorus extract and a Dipsacus asperoides extract with improved secretion capacity of IGF-1 and improved bone structure growth promotion capacity.

Another object of the present invention is to provide a Pleuropterus multiflorus extract and a Dipsacus asperoides extract manufactured by the method described above including glycoside components derived therefrom as an active ingredient.

A further object of the present invention is to provide foods and pharmaceutical products containing the Pleuropterus multiflorus extract and the Dipsacus asperoides extract.

### Technical Solution

In order to accomplish the above objects, the present invention provides a method for manufacturing a Pleuropterus multiflorus extract including: performing an extraction of Pleuropterus multiflorus; fermenting the Pleuropterus multiflorus extract obtained from the extraction; purifying the fermented Pleuropterus multiflorus extract obtained from the fermentation; and concentrating the purified Pleuropterus multiflorus extract obtained from the purification.

In an embodiment of the method for manufacturing a Pleuropterus multiflorus extract of the present invention, the extraction may be preferably performed by mixing from 6 to 10 volumes of water, relative to the dry weight of Pleuropterus multiflorus, having a temperature ranging from 80 to 100° C., with Pleuropterus multiflorus; followed by extracting while stirring the mixture for from 6 to 10 hours.

In an embodiment of the method for manufacturing a Pleuropterus multiflorus extract of the present invention, in order to increase the content of Pleuropterus multiflorus-derived glycoside, the fermentation may be preferably performed by adding from 1 to 5 wt % of α-amylase relative to the dry weight of Pleuropterus multiflorus to the Pleuropterus multiflorus extract, and fermenting the mixture at from 30 to 80° C. for from 2 to 24 hours.

In an embodiment of the method for manufacturing a Pleuropterus multiflorus extract of the present invention, the method, upon completion of the fermentation, may preferably further include terminating the activity of α-amylase by increasing the temperature of the fermented Pleuropterus multiflorus extract to 95° C. or higher, followed by quick freezing.

In an embodiment of the method for manufacturing a Pleuropterus multiflorus extract of the present invention, the purification may preferably include a first purification for removing insoluble microparticles and proteins from the Pleuropterus multiflorus extract by separating solids from the Pleuropterus multiflorus extract using a centrifugal hydro-extractor or a filter press, and subjecting them to ultracentrifugation at a rate ranging from 15,000 to 20,000 rpm.

In an embodiment of the method for manufacturing a Pleuropterus multiflorus extract of the present invention, the purification may preferably include a second purification for removing fat components, inactive organic compounds, tannin components, and heavy metals by passing the Pleuropterus multiflorus extract, which has been purified by the first purification, through an activated carbon filter.

In an embodiment of the method for manufacturing a Pleuropterus multiflorus extract of the present invention, the purification may preferably include a third purification process for removing high molecular carbohydrates and microorganisms from the Pleuropterus multiflorus extract, which has already undergone the second purification; which includes separating a retentate and a permeate from the Pleuropterus multiflorus extract via a Tangential Flow Filtration (TFF) type micro or ultra filtration system having a from 0.2 to 0.45 μm thick membrane, wherein the permeate is recovered therefrom but, when the flow rate drastically decreases due to a substantial increase in the filtration pressure caused by the increase in the retentate concentration, the retentate is diluted with purified water and is filtered several times repeatedly, preferably from 2 to 9 times.

In an embodiment of the method for manufacturing a Pleuropterus multiflorus extract of the present invention, the concentration may be preferably performed by adding the purified Pleuropterus multiflorus extract into a rotary evaporator and reducing the pressure at a temperature ranging from 40 to 80° C. until the brix reaches 10 or higher.

In an embodiment of the method for manufacturing a Pleuropterus multiflorus extract of the present invention, the method, upon completion of the concentration, may further include adding the concentrated Pleuropterus multiflorus extract into a lyophilizer and subjecting it to a stepwise temperature increased lyophilization cycle at a temperature ranging from −60 to 40° C. for from 30 to 40 hours, thereby obtaining a powdered Pleuropterus multiflorus extract.

Additionally, the present invention provides a Pleuropterus multiflorus extract manufactured according to the method described above, and is capable of promoting the secretion of insulin-like growth factor and bone structure growth; and includes a Pleuropterus multiflorus-derived saponin glycoside as an active ingredient.

Additionally, the present invention also provides foods and pharmaceutical products containing the Pleuropterus multiflorus extract described above.

The present invention provides a method for manufacturing a Dipsacus asperoides extract including: performing an extraction of Dipsacus asperoides; fermenting the Dipsacus asperoides extract obtained from the extraction; purifying the fermented Dipsacus asperoides extract obtained from the fermentation; and concentrating the purified Dipsacus asperoides extract obtained from the purification.

In an embodiment of the method for manufacturing a Dipsacus asperoides extract of the present invention, the extraction may be preferably performed by mixing from 6 to 10 volumes of water, relative to the dry weight of Dipsacus asperoides, and having a temperature ranging from 80 to 100° C., with Dipsacus asperoides; followed by extracting while stirring the mixture for from 6 to 10 hours.

In an embodiment of the method for manufacturing a Dipsacus asperoides extract of the present invention, the fermentation may be preferably performed to increase the content of Dipsacus asperoides-derived glycoside by adding from 1 to 5 wt % of α-amylase relative to the dry weight of Dipsacus asperoides to the Dipsacus asperoides extract, and fermenting the mixture at from 30 to 80° C. for from 2 to 24 hours.

In an embodiment of the method for manufacturing a Dipsacus asperoides extract of the present invention, the method, upon completion of the fermentation, may preferably further include terminating the activity of α-amylase by increasing the temperature of the fermented Dipsacus asperoides extract to 95° C. or higher, followed by quick freezing.

In an embodiment of the method for manufacturing a Dipsacus asperoides of the present invention, the purification may preferably include a first purification for removing insoluble microparticles and proteins from the Dipsacus asperoides extract by separating solids from the Dipsacus asperoides extract using a centrifugal hydro-extractor or a filter press, and subjecting them to ultracentrifugation at a rate ranging from 15,000 to 20,000 rpm.

In an embodiment of the method for manufacturing a Dipsacus asperoides extract of the present invention, the purification may preferably include a second purification for removing fat components, inactive organic compounds, tannin components, and heavy metals by passing the Dipsacus asperoides extract, which has previously been purified by the first purification, through an activated carbon filter.

In an embodiment of the method for manufacturing a Dipsacus asperoides extract of the present invention, the purification may preferably include a third purification for removing high molecular carbohydrates and microorganisms from the Dipsacus asperoides extract, which has been purified by the second purification, which includes separating a retentate and a permeate from the Dipsacus asperoides extract via a Tangential Flow Filtration (TFF) type micro or ultra filtration system having a from 0.2 to 0.45 μm thick membrane, wherein the permeate is recovered therefrom but, when the flow rate drastically decreases due to a substantial increase in the filtration pressure caused by the increase in the retentate concentration, the retentate is diluted with purified water and filtered several times repeatedly, preferably from 2 to 9 times.

In an embodiment of the method for manufacturing a Dipsacus asperoides extract of the present invention, the concentration may be preferably performed by adding the purified Dipsacus asperoides extract into a rotary evaporator and reducing the pressure at a temperature ranging from 40 to 80° C. until the brix reaches 10 or higher.

In an embodiment of the method for manufacturing a Dipsacus asperoides extract of the present invention, the method, upon completion of the concentration, may further include adding the concentrated Dipsacus asperoides extract into a lyophilizer and subjecting it to a stepwise temperature increased lyophilization cycle at a temperature ranging from −60 to 40° C. for from 30 to 40 hours, thereby obtaining a powdered Dipsacus asperoides extract.

Additionally, the present invention provides a Dipsacus asperoides extract, which is manufactured according to the method described above, and is capable of promoting the secretion of insulin-like growth factor and bone structure growth; and includes a Dipsacus asperoides-derived iridoid glycoside as an active ingredient.

Additionally, the present invention also provides foods and pharmaceutical products containing the Dipsacus asperoides extract described above.

### Advantageous Effects

The present invention provides a processing technology for mass production of aqueous extracts containing glycosides derived from Pleuropterus multiflorus and Dipsacus asperoides as an active ingredient. The aqueous glycoside extracts of Pleuropterus multiflorus and Dipsacus asperoides manufactured by the processing technology developed in the present invention have much improved effects in increasing the capacity of IGF-1 secretion and promoting bone structure growth as compared with those of the conventional technologies.

The aqueous extracts containing as active ingredients: glycoside components, purified after separation from Pleuropterus multiflorus and Dipsacus asperoides; can increase the secretion of IGF-1 thereby promoting bone structure growth, can be orally administered with ease, are cheaper than the therapeutic growth hormones manufactured via recombinant technology, and, as a non-toxic substance to humans promoting growth hormone secretion, can be used in manufacturing foods and pharmaceutical products.

From an industrial point of view, growth hormone has been of much interest for the past decade due to its biological activities and useful effects within the human body. According to the bio-pharmacological news released on the internet, the global market size of a human growth hormone injection (a recombinant protein produced by a microorganism) reached US $1.5 million in 2000, and has reportedly shown a rapid annual increase of 20% or higher. Recently, functional foods referred to as “growth hormone secretagogues” have been developed and are being sold in the U.S. by numerous health food companies. The major components of the products are mostly functional amino acids and vitamins that can enhance physical conditions of a body. In particular, a few of them contain an animal growth hormone releasing hormone (GHRH) therein because they are manufactured by directly adding a porcine pituitary gland extract. However, most people do not want to eat any extract obtained from animal tissues and no biological safeguard has been established against the risk of viral infection in handling the animal derived materials.

To develop a safe natural growth hormone secretagogue with biological activities to resolve the above problems, the inventors of the present invention searched through the natural plants disclosed in “Oriental Medicine”, and as a result, found that the extracts of Pleuropterus multiflorus and Dipsacus asperoides containing aqueous glycoside components derived therefrom as major components, have an excellent effect of increasing growth hormone release, which was confirmed by an animal experiment on growth promotion capacity using a hind leg femur mouse bone structure. Accordingly, it is speculated that the aqueous glycoside extracts of Pleuropterus multiflorus and Dipsacus asperoides provided in the present invention are essential core materials that may enter into the gigantic global market of growth hormone secretagogues; and they are highly likely to be developed into functional foods and beverages, as well as pharmaceutical products having a greater safety profile.

### DESCRIPTION OF DRAWINGS

FIG. 1 is a flowchart showing the mechanism of promoting the secretion of an insulin-like growth factor 1 (IGF-1) in a living subject by the glycoside components in plants.

FIG. 2 is a graph showing the comparison results of the contents of Pleuropterus multiflorus-derived glycosides with or without enzyme treatment.

FIG. 3 is a graph showing the comparison results of the contents of Dipsacus asperoides-derived glycosides in the presence or absence of an enzyme treatment.

FIG. 4 is a graph showing the changes in IGF-1 concentration according to time passage after administering the Pleuropterus multiflorus extracts prepared in Example 1 and Comparative Example 1, respectively.

FIG. 5 is a graph showing the changes in IGF-1 concentration according to time passage after administering Dipsacus asperoides extracts prepared in Example 2 and Comparative Example 2, respectively.

FIG. 6 is a graph showing the comparison results of the femur growth in a mouse after administering for 8 weeks the Pleuropterus multiflorus extracts prepared in Example 1 and Comparative Example 1, respectively.

FIG. 7 is a graph showing the comparison results of the femur growth in a mouse after administering for 8 weeks the Dipsacus asperoides extracts prepared in Example 2 and Comparative Example 2, respectively.

### MODE FOR INVENTION

The present invention is described in further details herein below.

The present invention relates to an aqueous purified extract containing as active ingredients glycoside components of Pleuropterus multiflorus and Dipsacus asperoides capable of increasing the capacity of secreting an insulin-like growth factor and promoting bone structure growth, and a method of manufacturing the same.

The term extract, as used herein, has a collective meaning referring to the various liquids generated during each respective step of the extraction process, including the original solution of the extract, a fermentation solution, a purified solution, a concentrate, and the powders in a solid form. In other words, all the substances produced in the course of the extraction process will be collectively called as an extract herein below.

While reviewing more than 30 natural herbal medicines that may be used as raw materials for foods, as selected from the numerous medicinal herbs with high growth-promoting potency registered in “Oriental Medicine”, including Crataegus pinnatifida var. typica, Saururus chinensis, cassia bark, Zingiberis officinale ROSC, Schisandra chinensis, morus bark, Perilla frutescens var. aceta, Paeonia lactiflora, Platycodi Radix, Chrysanthemum indicum L., Cinnamomum cassia Blume. Pleuropterus multiflorus, Polygala radix, Torilis japonica, Phyllostachys nigra var henonis, Acori gramineus, Prunus mume Sieb. et Zucc., Rubus coreanus Miguel, Dipsacus asperoides, lily, Bupleurum falcatum, Amomum villosum LOUR.), nutmeg, Thuja orientalis, myrrh, Acanthopanax, Mulberry Leaf Tea, Castanea crenata Leaf Tea and Cordyceps militaris, etc., the inventors of the present invention learned that the extracts of Pleuropterus multiflorus and Dipsacus asperoides have good efficacies in rectifying the capacity of secreting IGF-1 and promoting bone structure growth. As a result, the inventors pursued their research by further focusing their studies on the extracts of Pleuropterus multiflorus and Dipsacus asperoides and successfully provided a method to increase the contents of their active ingredients.

Pleuropterus multiflorus is a creeping perennial plant belonging to the Asclepiadaceae family and is known as an effective herbal medicine for health and longevity that darkens gray hairs. Pleuropterus multiflorus contains 1.8% of oxymetal anthraquinone derivative, 45% of carbohydrate, 3% of cynanchol as an essential oil, 3.7% of lecithin, a protein simultaneously having rhapontin and phosphatidylcholine as a hydrophilic group and a hydrophobic group, respectively, etc. In the present invention, a triterpene-based saponin as a cardiac glycoside among the components contained in Pleuropterus multiflorus was selected as a main component capable of regulating IGF-1 secretion capacity and promoting bone structure growth.

Dipsacus asperoides is a perennial plant belonging to the Labiatae family, and is known to control bones and sinews and blood vessels by healing broken bones and strengthening liver and kidney. Dipsacus asperoides contains alkaloid, essential oil, vitamins, etc. In the present invention, a shanzhigenin methyl ester as an iridoid glycoside among the components contained in Dipsacus asperoides was selected as a main component capable of increasing IGF-1 secretion capacity and promoting bone structure growth.

A glycoside refers to a binding between different components of a plant in which an active organic compound is bound to a sugar, for example, saponin+sugar, flavonoid+sugar, carotenoid+sugar, etc. It is a material produced by dehydrating condensation between a reducing group of a sugar and a hydroxyl group of another sugar or compound. Being present in all plants, it is involved in sugar storage, osmosis control, detoxification, and removal of wastes produced during plant metabolism.

The present invention provides a processing technology for mass production of aqueous extracts containing glycosides derived from Pleuropterus multiflorus and Dipsacus asperoides as an active ingredient. The aqueous glycoside extracts of Pleuropterus multiflorus and Dipsacus asperoides manufactured by the processing technology developed in the present invention was confirmed of their effects in increasing the secretion capacity of IGF-1 and promoting bone structure growth.

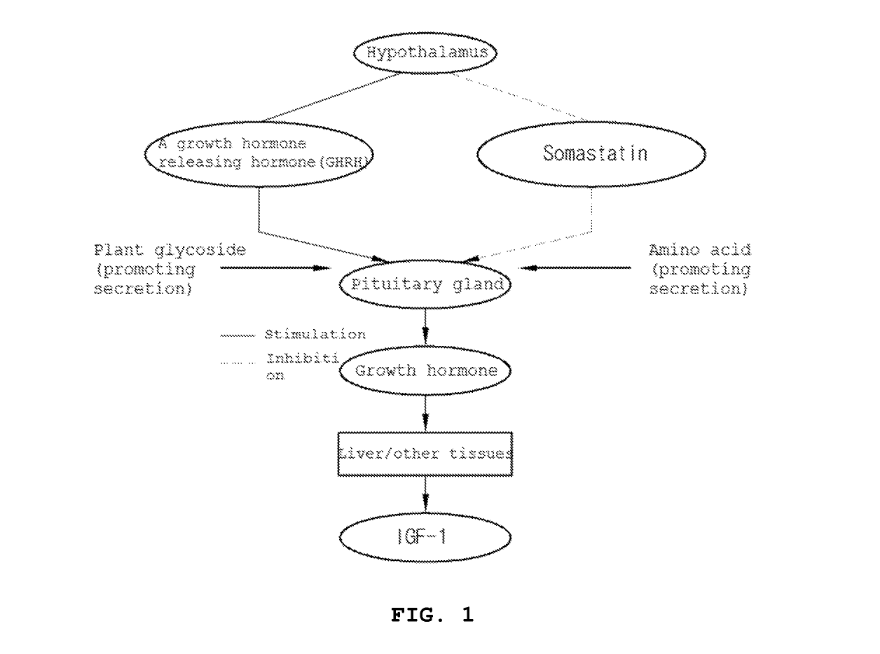

FIG. 1 is a flowchart showing the mechanism of promoting the secretion of an insulin-like growth factor 1 (IGF-1) in a living subject by the glycoside components in plants. The plant glycoside of the present invention can stimulate the pituitary gland thereby promoting the secretion of IGF-1.

The aqueous extracts containing as active ingredients glycoside components, purified after separation from Pleuropterus multiflorus and Dipsacus asperoides, can increase the secretion capacity of IGF-1 and promoting bone structure growth, can be orally administered with ease, are cheaper than the therapeutic growth hormones manufactured via recombinant technology, and, as a non-toxic substance to humans promoting growth hormone secretion, can be used in manufacturing foods and pharmaceutical products.

The method of manufacturing an aqueous extract containing Pleuropterus multiflorus-derived glycosides as active ingredients according to an embodiment of the present invention is explained in details herein below.

The method of manufacturing a Pleuropterus multiflorus extract according to an embodiment of the present invention may roughly include a processing, an extraction, a fermentation, a first purification, a second purification, a third purification, a concentration, and a lyophilization.

First, dry Pleuropterus multiflorus is processed into powders or slices in a size ranging from 2 to 5 mm in length.

Then, an extraction is performed under conditions suitable for the properties of Pleuropterus multiflorus-derived glycoside components. Hot water is preferably used as an extraction solvent. Extraction may be performed, for example, in an extraction tank equipped with a jacket, etc. More specifically, from 6 to 10 volumes of water, having a temperature ranging from 80 to 100° C., relative to the dry weight (L/kg) of Pleuropterus multiflorus is mixed with processed Pleuropterus multiflorus, and stirred for from 6 to 10 hours to obtain an aqueous Pleuropterus multiflorus extract, and the extract is cooled to 70° C. Due to the presence of a fermentation process, the present invention may shorten the extraction time.

Then, the Pleuropterus multiflorus extract is subjected to fermentation using an enzyme. Preferably, α-amylase, a sugar decomposing enzyme, effective in glycoside production may be used. The fermentation may be performed in the extraction tank used during the extraction or in an additionally prepared fermentation apparatus. The content of Pleuropterus multiflorus-derived glycosides may be increased through the fermentation process.

Specifically, from 1 to 5 wt % of α-amylase relative to the dry weight of Pleuropterus multiflorus is added into an extraction tank in which the Pleuropterus multiflorus extract is contained therein, and the mixture is subjected to fermentation at a temperature ranging from 30 to 80° C. for from 2 to 24 hours, thereby fermenting the Pleuropterus multiflorus extract. Here, when the amount of the enzyme used is too little the resulting fermentation products such as glycosides, etc., become small thus making the fermentation effect negligible. On the other hand, when the amount of the enzyme used is excessive it may increase the production cost relative to the fermentation effect. When the fermentation temperature is too low it reduces the fermentation speed thus increasing the fermentation time, whereas when the fermentation temperature is too high functional components may be destroyed. When the fermentation time is too short the fermentation effect may be negligible, whereas when the fermentation time is too long it may increase the production cost relative to the fermentation effect.

As such, the contents of natural substances-derived glycosides may be significantly increased by adding an appropriate enzyme in an effective amount, and conducting the fermentation in an appropriate temperature and time for fermentation. Accordingly, the effects of rectifying the capacity of insulin-like growth factor and promoting the bone structure growth may be considerably improved.

Then, upon completion of the fermentation of the Pleuropterus multiflorus extract, the fermented extract is increased to 95° C. or higher, and then immediately cooled by adding a coolant inside the jacket of the extraction tank, thereby terminating the activity of α-amylase.

Then, in order to remove solids from the aqueous fermented extract of Pleuropterus multiflorus, the solids were separated using a centrifugal hydro-extractor or a filter press, and insoluble microparticles and proteins were removed by subjecting the aqueous fermented extract of Pleuropterus multiflorus to a first purification via ultracentrifugation at a rate ranging from 15,000 to 20,000 rpm, thereby obtaining an aqueous Pleuropterus multiflorus-derived first purified liquid in a transparent color.

Then, in order to remove fat components, inactive organic compounds, tannin components, heavy metals, etc., contained in the aqueous Pleuropterus multiflorus-derived first purification liquid obtained from the centrifugation, the aqueous Pleuropterus multiflorus-derived first purification liquid was passed through an activated carbon filter as a second purification.

Then, in order to remove inactive high molecular carbohydrates and microorganisms in the aqueous Pleuropterus multiflorus-derived second purification liquid obtained by activated carbon filtration, the aqueous Pleuropterus multiflorus-derived second purification liquid was separated into a retentate and a permeate using a TFF type micro/ultra filtration system equipped with a 0.2 to 0.45 μm thick membrane capable of a continuous filtration without clogging during filtration, and then only the aqueous Pleuropterus multiflorus-derived third purification liquid as a permeate was recovered. Here, in order to maximally obtain the glycoside components of Pleuropterus multiflorus in the second purification liquid being retained, when the flow rate drastically decreases due to a substantial increase in the filtration pressure caused by the increase in the retentate concentration, the retentate is diluted with purified water and repeatedly filtered a few times (preferably from 2 to 9 times).

Then, the resulting aqueous Pleuropterus multiflorus-derived third purification liquid, purified after a few times of repeated filtration (preferably from 2 to 9 times), is concentrated by adding it into a rotary evaporator and reducing the pressure at a temperature ranging from 40 to 60° C. until the brix reaches 10 or higher, thereby finally obtaining an aqueous purified liquid of glycoside extract derived from Pleuropterus multiflorus.

Then, the concentrate is added into a lyophilizer, and subjected to a stepwise temperature increased lyophilization cycle at a temperature ranging from −60 to 40° C. for from 30 to 40 hours, thereby obtaining an aqueous purified powder of glycoside extract derived from Pleuropterus multiflorus. The lyophilization may be selectively performed upon necessity.

The present invention provides a Pleuropterus multiflorus extract manufactured by the method described above, which has the capacities of promoting the secretion of insulin-like growth factor and the bone structure growth, and contains a Pleuropterus multiflorus-derived saponin glycoside as an active ingredient.

The Pleuropterus multiflorus extract of the present invention may be used to manufacture foods and pharmaceutical products.

When the Pleuropterus multiflorus extract is used in the pharmaceutical products, additives such as a carrier, an excipient, a lubricant, a wetting agent, a sweetener, a flavor, an emulsifier, a suspension, a preservative, a dispersant and/or a stabilizer may be added upon necessity. The pharmaceutical products containing the Pleuropterus multiflorus extract may be formulated into a solution in oil or aqueous medium, a suspension or an emulsion, or an extract, powder, granules, a tablet, or a capsule. The appropriate dose of the pharmaceutical product may vary depending on factors such as the formulation method, administration method, the age, body weight, sex, severeness of a disease of a patient, diet, duration of administration, administration route, excretion rate and reaction sensitivity; and an experienced physician may easily determine and prescribe an effective dose for the intended treatment or prevention. For example, in the case of oral administration, the Pleuropterus multiflorus extract may be administered to an adult once daily in the amount ranging from 0.3 to 3 g. It may be administered orally or parenterally, but preferably by oral administration.

The Pleuropterus multiflorus extract may be used in all kinds of foods including beverages, health foods, liquors, etc. For its use as a drink, for example, citric acid, high fructose corn syrup, sugar, glucose, acetic acid, malic acid, fruit juices, etc., may be added in addition to the active ingredient of the present invention.

In an another embodiment of the present invention, a method of manufacturing an aqueous extract containing Dipsacus asperoides-derived glycoside as an active ingredient is described in detail herein below.

The method of manufacturing a Dipsacus asperoides extract according to an embodiment of the present invention may roughly include a processing, an extraction, a fermentation, a first purification, a second purification, a third purification, a concentration, and a lyophilization.

First, dry Dipsacus asperoides is processed into powders or slices in a size ranging from 2 to 5 mm in length.

Then, an extraction is performed under conditions suitable for the properties of Dipsacus asperoides-derived glycoside components. More specifically, from 6 to 10 volumes of water, having a temperature ranging from 80 to 100° C., relative to the dry weight (L/kg) of Dipsacus asperoides is mixed with processed Dipsacus asperoides, and stirred for from 6 to 10 hours to obtain an aqueous Dipsacus asperoides extract, and the extract is cooled to 70° C.

Then, the Dipsacus asperoides extract is subjected to fermentation using an enzyme. Specifically, from 1 to 5 wt % of α-amylase relative to the dry weight of Dipsacus asperoides is added into an extraction tank in which the Dipsacus asperoides extract is contained therein, and the mixture is subjected to fermentation at a temperature ranging from 30 to 80° C. for from 2 to 24 hours, thereby fermenting the Dipsacus asperoides extract.

Then, upon completion of the fermentation of the Dipsacus asperoides extract, the fermented extract is increased to 95° C. or higher, and then immediately cooled by adding a coolant inside the jacket of the extraction tank, thereby terminating the activity of α-amylase.

Then, in order to remove solids from the aqueous fermented extract of Dipsacus asperoides, the solids were separated using a centrifugal hydro-extractor or a filter press, and insoluble microparticles and proteins were removed by subjecting the aqueous fermented extract of Dipsacus asperoides to a first purification via ultracentrifugation at a rate ranging from 15,000 to 20,000 rpm, thereby obtaining an aqueous Dipsacus asperoides-derived first purified liquid in a transparent, clear, colorless solution.

Then, in order to remove fat components, inactive organic compounds, tannin components, heavy metals, etc., contained in the aqueous Dipsacus asperoides-derived first purification liquid obtained from the centrifugation, the aqueous Dipsacus asperoides-derived first purification liquid was passed through an activated carbon filter as a second purification.

Then, in order to remove inactive high molecular carbohydrates and microorganisms in the aqueous Dipsacus asperoides-derived second purification liquid obtained by activated carbon filtration, the aqueous Dipsacus asperoides-derived second purification liquid was separated into a retentate and a permeate using a TFF type micro/ultra filtration system equipped with a from 0.2 to 0.45 μm thick membrane capable of a continuous filtration without clogging during filtration, and then only the aqueous Dipsacus asperoides-derived third purification liquid as a permeate was recovered. Here, in order to maximally obtain the glycoside components of Dipsacus asperoides in the second purification liquid being retained, when the flow rate drastically decreases due to a substantial increase in the filtration pressure caused by the increase in the retentate concentration, the retentate is diluted with purified water and is repeatedly filtered a few times (preferably from 2 to 9 times).

Then, the aqueous Dipsacus asperoides-derived third purification liquid, penetrated after a few times of repeated filtration (preferably from 2 to 9 times), is concentrated by adding it into a rotary evaporator and reducing the pressure at a temperature ranging from 40 to 60° C. until the brix reaches 10 or higher, thereby finally obtaining an aqueous purified liquid of glycoside extract derived from Dipsacus asperoides.

Then, the concentrate is added into a lyophilizer, and subjected to a stepwise temperature increased lyophilization cycle at a temperature ranging from −60 to 40° C. for from 30 to 40 hours, thereby obtaining an aqueous purified powder of glycoside extract derived from Dipsacus asperoides.

The present invention provides a Dipsacus asperoides extract manufactured by the method described above, which has the capacities of promoting the secretion of insulin-like growth factor and the bone structure growth, and contains a Dipsacus asperoides-derived iridoid glycoside as an active ingredient.

The Dipsacus asperoides extract of the present invention may be used to manufacture foods and pharmaceutical products.

A better understanding of the present invention regarding its invention features and effects may be obtained through the following examples which are set forth to illustrate, but are not to be construed as the limit of the present invention.

### Example 1

First, dry Pleuropterus multiflorus was processed into powders or slices in a size of 4 mm in length.

Then, about 8 volumes of water at 90° C. relative to the weight of dry Pleuropterus multiflorus was mixed with processed Pleuropterus multiflorus in an extraction tank equipped with a jacket and stirred for 8 hours and obtained an aqueous extract of Pleuropterus multiflorus, which was then cooled to 70° C.

Then, 3 wt % of α-amylase relative to the weight of dry Pleuropterus multiflorus was added into an extraction tank in which the Pleuropterus multiflorus extract was contained, and the mixture was fermented at 70° C. for 6 hours.

Then, upon completion of the fermentation of the Pleuropterus multiflorus extract, the fermented extract was increased to 95° C. or higher, and then immediately cooled by adding a coolant inside the jacket of the extraction tank, thereby terminating the activity of α-amylase.

Then, the solids were separated from the aqueous fermented extract of Pleuropterus multiflorus using a centrifugal hydro-extractor or a filter press and then ultracentrifuged at a rate ranging from 18,000 rpm, thereby obtaining an aqueous Pleuropterus multiflorus-derived first purified liquid.

Then, the first purified liquid was passed through an activated carbon filter to obtain a second purified liquid.

Then, the second purified liquid was separated by a TFF type micro/ultra filtration system equipped with a 0.45 μm thick membrane, and then only the aqueous Pleuropterus multiflorus-derived third purification liquid as a permeate was recovered. When the flow rate drastically decreased due to a substantial increase in the filtration pressure caused by the increase in the retentate concentration, the retentate was diluted with purified water and repeatedly filtered a few times (preferably from 2 to 9 times).

Then, the aqueous Pleuropterus multiflorus-derived third purification liquid was concentrated by adding it into a rotary evaporator and reducing the pressure at 60° C. until the brix reached 10 or higher, thereby finally obtaining a concentrate.

Finally, the concentrate was added into a lyophilizer, and subjected to a stepwise temperature increased lyophilization cycle at a temperature ranging from −60 to 40° C. for 36 hours, and obtained an aqueous purified powder of glycoside extract derived from Pleuropterus multiflorus.

### Example 2

First, dry Dipsacus asperoides was processed into powders or slices in a size of 4 mm in length.

Then, about 8 volumes of water at 90° C. relative to the weight of dry Dipsacus asperoides was mixed with processed Dipsacus asperoides in an extraction tank equipped with a jacket and stirred for 8 hours and obtained an aqueous extract of Dipsacus asperoides, which was then cooled to 70° C.

Then, 3 wt % of α-amylase relative to the weight of dry Dipsacus asperoides was added into an extraction tank in which the Dipsacus asperoides extract was contained, and the mixture was fermented at 70° C. for 6 hours.

Then, upon completion of the fermentation of the Dipsacus asperoides extract, the fermented extract was increased to 95° C. or higher, and then immediately cooled by adding a coolant inside the jacket of the extraction tank, thereby terminating the activity of α-amylase.

Then, the solids were separated from the aqueous fermented extract of Dipsacus asperoides using a centrifugal hydro-extractor or a filter press and then ultracentrifuged at a rate ranging from 18,000 rpm, thereby obtaining an aqueous Dipsacus asperoides-derived first purified liquid.

Then, the first purified liquid was passed through an activated carbon filter to obtain a second purified liquid.

Then, the second purified liquid was separated by a TFF type micro/ultra filtration system equipped with a 0.45 μm thick membrane, and then only the aqueous Dipsacus asperoides-derived third purification liquid as a permeate was recovered. When the flow rate drastically decreased due to a substantial increase in the filtration pressure caused by the increase in the retentate concentration, the retentate was diluted with purified water and repeatedly filtered a few times (preferably from 2 to 9 times).

Then, the aqueous Dipsacus asperoides-derived third purification liquid was concentrated by adding it into a rotary evaporator and reducing the pressure at 60° C. until the brix reached 10 or higher, thereby finally obtaining a concentrate.

Finally, the concentrate was added into a lyophilizer, and subjected to a stepwise temperature increased lyophilization cycle at a temperature ranging from −60 to 40° C. for 36 hours, and obtained an aqueous purified powder of glycoside extract derived from Dipsacus asperoides.

### Comparative Example 1

The experiment was performed in the same manner as in Example 1 except that fermentation was not performed.

### Comparative Example 2

The experiment was performed in the same manner as in Example 2 except that fermentation was not performed.

### Test Example 1

In Test Example 1, the glycoside contents present in Pleuropterus multiflorus extract and Dipsacus asperoides extract with or without treatment of α-amylase were compared.

The test method is as follows. First, 1 g each of samples prepared in Examples and Comparative Examples were dissolved in 60 mL of distilled water, transferred into a separatory funnel, and extracted with 60 mL of ether.

After adding 60 mL of water-saturated butanol into the separatory funnel, the funnel was shaken and the resulting top layer was recovered. The entire process was repeated 3 times.

Subsequently, all the recovered butanol layers were combined and washed with 50 mL of distilled water, and the top layer was recovered.

Then, the recovered solution was added into a flask with a known weight, concentrated under reduced pressure in an evaporator at 80° C., and dried in a drying oven at 105° C. for 15 minutes.

The resultant was completely cooled within a desiccators and weighed.

The contents of glycosides were calculated using the equation shown below.

glycoside content=(flask weight after concentration−flask weight before concentration)/amount of sample (g)×100

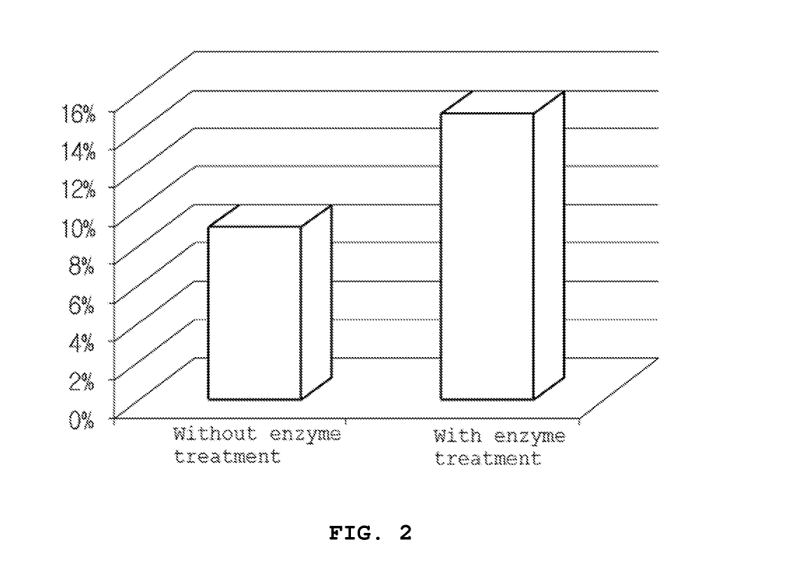

Table 1 shows the results of experiments comparing the contents of Pleuropterus multiflorus-derived prepared in Example 1 and Comparative Example 1. FIG. 2 is a graph showing the comparison results of the contents of Pleuropterus multiflorus-derived glycosides with or without enzyme treatment.

TABLE 1

| Category (average of 3 | Ratio of glycoside | Ratio of glycoside |
|---|---|---|
| experiments) | contents (%) | contents (mg/g) |
| C. Ex. 1 (without | 8.95 | 89.5 |
| enzyme treatment) | | |
| Ex. 1 (with enzyme | 14.87 | 148.7 |
| treatment) | | |

As can be seen from the results of Table 1 and FIG. 2, the glycoside content as a target component was higher in Example 1, where the fermentation was performed in the presence of α-amylase, by 66% than that in Comparative Example 1, where the fermentation was performed without α-amylase.

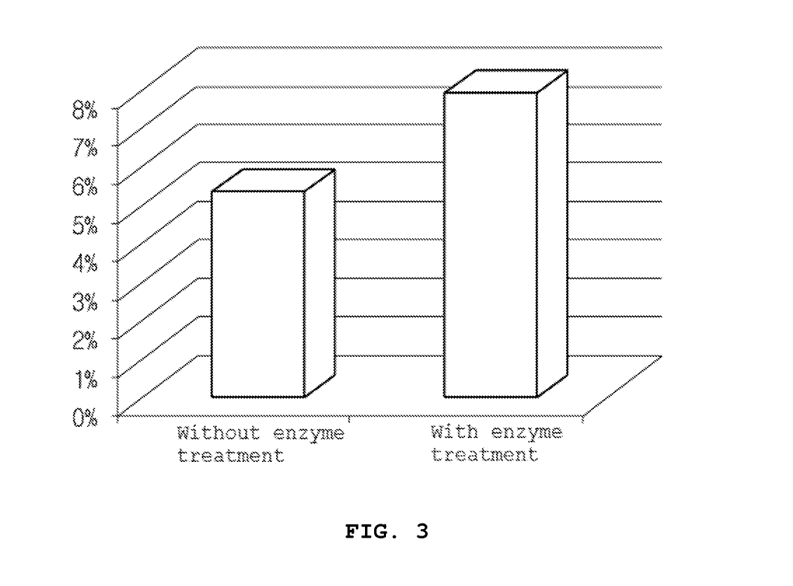

Table 2 shows the results of experiments comparing the contents of Dipsacus asperoides-derived glycosides prepared in Example 2 and Comparative Example 2. FIG. 3 is a graph showing the comparison results of the contents of Dipsacus asperoides-derived with or without enzyme treatment.

TABLE 2

| Category (average of 3 | Ratio of glycoside | Ratio of glycoside |
|---|---|---|
| experiments) | contents (%) | contents (mg/g) |
| C. Ex. 2 (without | 5.32 | 53.2 |
| enzyme treatment) | | |
| Ex. 2 (with enzyme | 7.98 | 79.8 |
| treatment) | | |

As can be seen from the results of Table 2 and FIG. 3, the glycoside content as a target component was higher in Example 2, where the fermentation was performed in the presence of α-amylase, by 50% than that in Comparative Example 2, where the fermentation was performed without α-amylase.

### Test Example 2

In Test Example 2, the glycoside aqueous extracts, derived from Pleuropterus multiflorus and Dipsacus asperoides, prepared in Examples and Comparative Examples were tested for their capacities of promoting IGF-1 secretion and bone structure growth.

The devices used for the analyses were ELISA reader (LAB SYSTEM, USA) and ELISA kit (rat IGF-1, catalogue # DSL 10-2900, Diagnostic Systems Laboratories, USA). The system used for data analyses and statistics was Prism (ver. 2.01, Graphpad Software Inc, USA).

Animals used for the experiments were Sprague Dawley rats. The 3 week old experimental animals were used for the experiments with long term administration and 9 week old ones were used for IGF-1 evaluation. All experimental animals were males, and 30 rats were alloted per each experiment (10 for control group, 10 for Example, and 10 for Comparative Example). The rats were raised under the conditions set at 22±3(19-25°) C. with 30-70% humidity and photoperiod (12 hours, light cycle: 08:00-20:00).

For the evaluation of IGF-1, in order to regulate the basic amount of growth hormone in the blood or for simultaneity purpose, all animals were used after being starved for 24 hours. Rats in test groups in Examples and Comparative Examples were orally administered of a daily dose based on the weight comparison between humans and rats (1,500 mg daily per person with 60 kg). Rats in the control group were fed with an equal volume of drinks to that in the test group. Blood samples were collected from the tales at time 0, and after oral administration, were continuously collected at 2 hour intervals until 10 hours after the administration. Blood sera were separated from the samples to prepare samples for evaluation. The level of IGF-1 was measured via ELISA using a kit.

For the test of long term administration, the extracts prepared in Examples and Comparative Examples in the amount of a daily dose were administered after mixing it with a feed based on the weight comparison between humans and rats (1,500 mg daily per person with 60 kg). The rats in the control group were fed with normal feeds, and the amount of the feed intake and the drinks were confirmed to be the same. The feeds and drinks were supplied fresh via daily replacement, and provided continuously for 8 consecutive weeks. The presence of any changes in femur bones was observed during the test.

Since most growth hormones are instantly released by a pulsed mode it is difficult to measure the change in the amount of growth hormone release in response to a physiological stimulus. Although hormones generally flow through the bloodstream for only a few minutes, the duration is sufficient to stimulate conversion into a growth factor in the liver once the hormones reach the liver. The secretion of IGF-1 may be used to measure the amount of growth hormone release. In fact, IGF-1 is involved in more versatile roles than growth hormone itself, and is also directly involved in most biological activities. Accordingly, in the experiment, the amount of IGF-1 secretion in blood, which is a secondary signal of growth hormone, was measured. IGF-1 is stably maintained in the blood upon receipt of a stimulus, and substantially exhibits the effects of growth hormone.

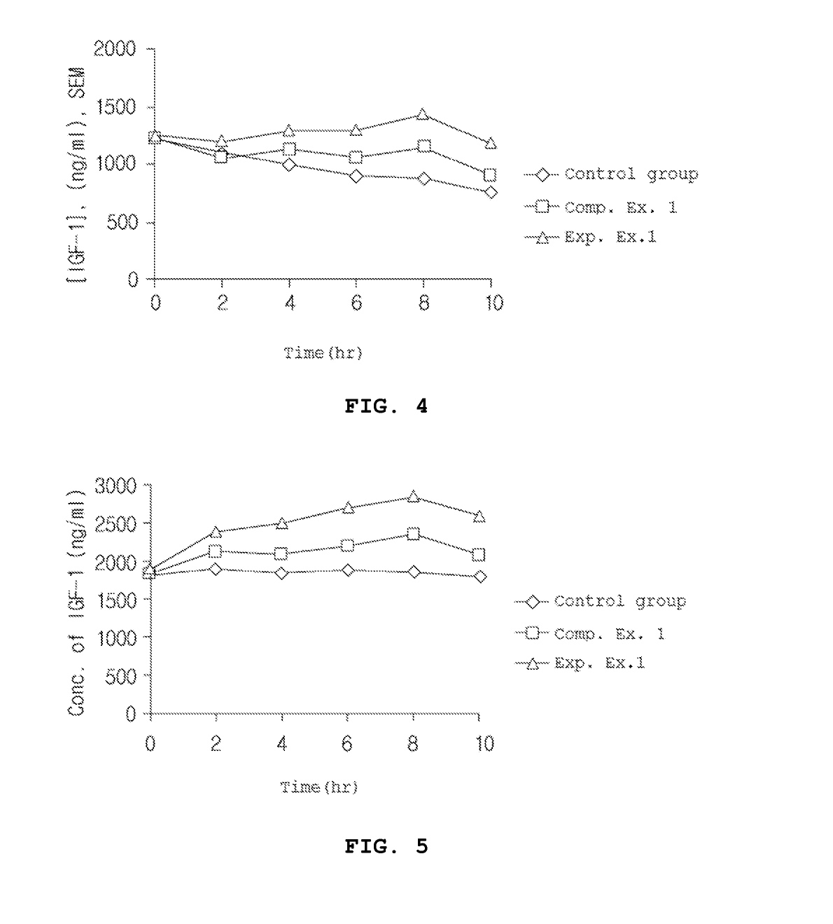

According to the IGF-1 evaluation test, the evaluation results of the effects of aqueous glycoside extracts derived from Pleuropterus multiflorus and Dipsacus asperoides on promoting secretion of growth hormone are shown in FIG. 4 and FIG. 5, respectively.

FIG. 4 is a graph showing the changes in IGF-1 concentration according to time passage after administering the Pleuropterus multiflorus extracts prepared in Example 1 and Comparative Example 1, respectively; and FIG. 5 is a graph showing the changes in IGF-1 concentration according to time passage after administering Dipsacus asperoides extracts prepared in Example 2 and Comparative Example 2, respectively.

As can be seen in FIG. 4, the amount of IGF-1 secretion continuously decreased in the control group but that in Comparative Example 1 was maintained overall, and that in Example 1 was increased significantly. Based on the time point of 8 hours after administration, where the maximum value was shown, the amount of IGF-1 secretion in Example 1 was greater than that in the control group by about 64%, and by about 24% than that in the Comparative Example 1. Accordingly, the Pleuropterus multiflorus extract manufactured according to the method of the present invention including a fermentation process was shown to have excellent effect on promoting the secretion of IGF-1.

As can be seen in FIG. 5, the amount of IGF-1 secretion in the control group was maintained overall, whereas that in Comparative Example 2 was increased significantly, and that in Example 2 was increased even further. Based on the time point of 8 hours after administration, where the maximum value was shown, the amount of IGF-1 secretion in Example 2 was greater than that in the control group by about 53%, and by about 21% than that in Comparative Example 2. Accordingly, the Dipsacus asperoides extract manufactured according to the method of the present invention including a fermentation process was shown to have excellent effect on promoting the secretion of IGF-1.

Referring to FIG. 4 and FIG. 5, the level of IGF-1 secretion in Examples increased up to 8 hours after the administration and then decreased. The secretion mode suggested that the test material did not directly stimulate the liver to induce the secretion of IGF-1 into the blood. Furthermore, the IGF-1 secretion pattern in Examples agrees with the general characteristics of secretion of IGF-1, which is induced 6 to 12 hours after administration of growth hormone.

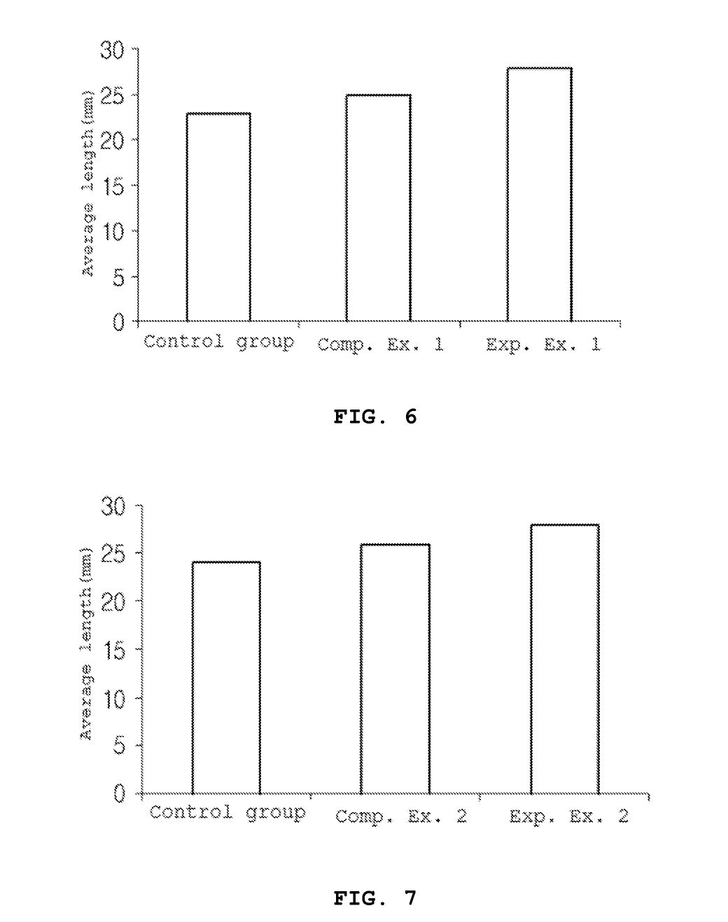

The effects of long term administration on promoting the bone structure growth are shown in FIG. 6 and FIG. 7.

FIG. 6 is a graph showing the comparison results of the femur growth in a mouse after administering for 8 weeks the Pleuropterus multiflorus extracts prepared in Example 1 and Comparative Example 1, respectively; and FIG. 7 is a graph showing the comparison results of the femur growth in a mouse after administering for 8 weeks the Dipsacus asperoides extracts prepared in Example 2 and Comparative Example 2, respectively.

As can be seen in FIG. 6, the average length of the rats' femur bones was about 22% longer than that in the control group, and about 12% longer than that in Comparative Example 1. Accordingly, the Pleuropterus multiflorus extract manufactured according to the method of the present invention including a fermentation process was shown to have excellent effect on promoting the bone structure growth.

As can be seen in FIG. 7, the average length of the rats' femur bones in Example 2 was longer by about 17% than that in the control group, and longer by about 8% than that in Comparative Example 2. Accordingly, the Dipsacus asperoides extract manufactured according to the method of the present invention including a fermentation process was shown to have excellent effect on promoting the bone structure growth.

Referring to the results shown in FIG. 4 to FIG. 7, it was confirmed that the increase in the amount of IGF-1 serves as an important factor in promoting the bone structure growth regarding the both aqueous glycoside extract of Pleuropterus multiflorus and Dipsacus asperoides.

Conclusively, two different kinds of experiments were performed in order to evaluate the biological effects of aqueous glycoside extracts of Pleuropterus multiflorus and Dipsacus asperoides on promoting secretion of growth hormone; i.e., the first experiment related to the effects of a short term administration and the change in the amount of IGF-1 secretion after a single administration, and the second experiment related to the effects of a long term administration and the change in bone structure growth was compared.

As can be seen in FIG. 4 and FIG. 5, when the changes in the amount of IGF-1 secretion in rats after a single administration of Pleuropterus multiflorus aqueous glycoside extract and Dipsacus asperoides aqueous glycoside extract were observed according to time passage, the amounts of IGF-1 secretion in Examples was significantly higher than those in the control group and Comparative Examples.

As can be seen in FIG. 6 and FIG. 7, when the length of the bone structure in the rats in Examples were longer than those in the control group and the Comparative Examples. The results confirmed that the extracts prepared in Examples still retained physiological activities even when they were orally administered. Besides, regarding the both aqueous glycoside extracts derived from Pleuropterus multiflorus and Dipsacus asperoides, it is speculated that the increase in the amount of IGF-1 secretion caused by oral administration is directly associated with the promotion on the bone structure growth in rats.

What is claimed is:

1. A Pleuropterus multiflorus extract for promoting the secretion of an insulin-like growth factor and bone structure growth, the method of its manufacture comprising:
(a) mixing from 6 to 10 volumes of water relative to the dry weight of Pleuropterus multiflorus, having a temperature ranging from 80 to 100° C., with Pleuropterus multiflorus, extracting while stirring the mixture for from 6 to 10 hours, followed by cooling the extract to 70° C.;
(b) increasing the content of Pleuropterus multiflorus-derived glycoside by adding 4 to 5 wt % of α-amylase relative to the dry weight of Pleuropterus multiflorus to the Pleuropterus multiflorus extract, and fermenting the mixture at from 70 to 80° C. for from 2 to 3 hours;
(c) upon completion of the fermentation, terminating the activity of α-amylase by increasing the temperature of the fermented Pleuropterus multiflorus extract to from 95 to 99° C., followed by quick freezing;
(d) performing a first purification for removing insoluble microparticles and proteins from the Pleuropterus multiflorus extract by separating solids from the Pleuropterus multiflorus extract using a centrifugal hydro-extractor or a filter press, and subjecting them to ultracentrifugation at a rate ranging from 15,000 to 20,000 rpm;
(e) performing a second purification for removing fat components, inactive organic compounds, tannin components, and heavy metals from by passing the Pleuropterus multiflorus extract, which has been purified by the first purification, through an activated carbon filter;
(f) performing a third purification for removing high molecular carbohydrates and microorganisms from the Pleuropterus multiflorus extract, which has been purified by the second purification, which includes separating a retentate and a permeate from the Pleuropterus multiflorus extract via a Tangential Flow Filtration (TFF) type micro or ultra filtration system having a 0.2 to 0.45 μm thick membrane, wherein the permeate is recovered therefrom but, when the flow rate drastically decreases due to a substantial increase in the filtration pressure caused by the increase in the retentate concentration, the retentate is diluted with purified water and repeatedly filtered from 2 to 9 times;
(g) concentrating the purified Pleuropterus multiflorus extract by adding it into a rotary evaporator and reducing the pressure at a temperature ranging from 40 to 60° C. until the brix reaches 10 or higher; and
(h) upon completion of concentration, further comprising adding the concentrated Pleuropterus multiflorus extract into a lyophilizer and subjecting it to a stepwise temperature increased lyophilization cycle at a temperature ranging from −60 to 40° C. for from 30 to 40 hours, thereby obtaining a powdered Pleuropterus multiflorus extract,
wherein the Pleuropterus multiflorus extract is capable of promoting the secretion of insulin-like growth factor and bone structure growth; and comprises a Pleuropterus multiflorus-derived saponin glycoside as an active ingredient.

2. A food containing the Pleuropterus multiflorus extract for promoting the secretion of an insulin-like growth factor and bone structure growth of claim 1.

3. A Dipsacus asperoides extract for promoting the secretion of an insulin-like growth factor and bone structure growth, the method of its manufacture comprising:
(a) mixing from 6 to 10 volumes of water, having a temperature ranging from 80 to 100° C., relative to the dry weight of Dipsacus asperoides with Dipsacus asperoides, extracting while stirring the mixture for from 6 to 10 hours, followed by cooling the extract to 70° C.;
(b) increasing the content of Dipsacus asperoides-derived glycoside by adding 4 to 5 wt % of α-amylase relative to the dry weight of Dipsacus asperoides to the Dipsacus asperoides extract, and fermenting the mixture at 70 to 80° C. for from 2 to 3 hours;
(c) upon completion of the fermentation, terminating the activity of α-amylase by increasing the temperature of the fermented Dipsacus asperoides extract to from 95 to 99° C., followed by quick freezing;
(d) performing a first purification for removing insoluble microparticles and proteins from the Dipsacus asperoides extract by separating solids from the Dipsacus asperoides extract using a centrifugal hydro-extractor or a filter press, and subjecting them to ultracentrifugation at a rate ranging from 15,000 to 20,000 rpm;
(e) performing a second purification for removing fat components, inactive organic compounds, tannin components, and heavy metals by passing the Dipsacus asperoides extract, which has been purified by the first purification, through an activated carbon filter;
(f) performing a third purification for removing high molecular carbohydrates and microorganisms from the Dipsacus asperoides extract, which has been purified by the second purification, which includes separating a retentate and a permeate from the Dipsacus asperoides extract via a Tangential Flow Filtration (TFF) type micro or ultra filtration system having a 0.2 to 0.45 μm thick membrane, wherein the permeate is recovered therefrom but, when the flow rate drastically decreases due to a substantial increase in the filtration pressure caused by the increase in the retentate concentration, the retentate is diluted with purified water and repeatedly filtered from 2 to 9 times;
(g) concentrating the purified Dipsacus asperoides extract by adding it into a rotary evaporator and reducing the pressure at a temperature ranging from 40 to 60° C. until the brix reaches 10 or higher; and
(h) upon completion of concentration, further comprising adding the concentrated Dipsacus asperoides extract into a lyophilizer and subjecting it to a stepwise temperature increased lyophilization cycle at a temperature ranging from −60 to 40° C. for 30 to 40 hours, thereby obtaining a powdered Dipsacus asperoides extract,
wherein the Dipsacus asperoides extract is capable of promoting the secretion of insulin-like growth factor and bone structure growth; and comprises a Dipsacus asperoides-derived iridoid glycoside as an active ingredient.

4. A food containing the Pleuropterus multiflorus extract for promoting the secretion of an insulin-like growth factor and bone structure growth of claim 3.
